(19) United States
(12) Patent Application Publication
(10) Pub. No.: US 2023/0280483 A1
(43) Pub. Date: Sep. 7, 2023

(54) IMAGING APPARATUS

(71) Applicant: SHENZHEN XPECTVISION TECHNOLOGY CO., LTD., Shenzhen (CN)

(72) Inventors: Yurun LIU, Shenzhen (CN); Peiyan CAO, Shenzhen (CN)

(21) Appl. No.: 18/195,998

(22) Filed: May 11, 2023

Related U.S. Application Data
(63) Continuation of application No. PCT/CN2020/131470, filed on Nov. 25, 2020.

(51) Int. Cl. G01T 1/29; G01T 1/15; G01T 1/36; G01T 1/17

(52) U.S. Cl. CPC G01T 1/2971; G01T 1/2992; G01T 1/15; G01T 1/366; G01T 1/17

(57) ABSTRACT

Disclosed herein is an apparatus comprising: a radiation detector; a collimator; wherein the collimator and the radiation detector are configured to collectively translate relative to a radiation source along a direction without relative movement the collimator and the radiation detector; wherein the collimator comprises a plurality of planar plates parallel to one another; and wherein the planar plates are not parallel to the direction.

### BACKGROUND

Radiation detectors may be devices used to measure the flux, spatial distribution, spectrum or other properties of radiations.

Radiation detectors may be used for many applications. One important application is imaging. Radiation imaging is a radiography technique and can be used to reveal the internal structure of a non-uniformly composed and opaque object such as the human body.

Early radiation detectors for imaging include photographic plates and photographic films. A photographic plate may be a glass plate with a coating of light-sensitive emulsion. Although photographic plates were replaced by photographic films, they may still be used in special situations due to the superior quality they offer and their extreme stability. A photographic film may be a plastic film (e.g., a strip or sheet) with a coating of light-sensitive emulsion.

In the 1980s, photostimulable phosphor plates (PSP plates) became available. A PSP plate may contain a phosphor material with color centers in its lattice. When the PSP plate is exposed to radiation, electrons excited by radiation are trapped in the color centers until they are stimulated by a laser beam scanning over the plate surface. As the plate is scanned by laser, trapped excited electrons give off light, which is collected by a photomultiplier tube. The collected light is converted into a digital image. In contrast to photographic plates and photographic films, PSP plates can be reused.

Another kind of radiation detectors are radiation image intensifiers. Components of a radiation image intensifier are usually sealed in a vacuum. In contrast to photographic plates, photographic films, and PSP plates, Radiation image intensifiers may produce real-time images, i.e., do not require post-exposure processing to produce images. Radiation first hits an input phosphor (e.g., cesium iodide) and is converted to visible light. The visible light then hits a photocathode (e.g., a thin metal layer containing cesium and antimony compounds) and causes emission of electrons. The number of emitted electrons is proportional to the intensity of the incident Radiation. The emitted electrons are projected, through electron optics, onto an output phosphor and cause the output phosphor to produce a visible-light image.

Scintillators operate somewhat similarly to radiation image intensifiers in that scintillators (e.g., sodium iodide) absorb radiation and emit visible light, which can then be detected by a suitable image sensor for visible light. In scintillators, the visible light spreads and scatters in all directions and thus reduces spatial resolution. Reducing the scintillator thickness helps to improve the spatial resolution but also reduces absorption of radiation. A scintillator thus has to strike a compromise between absorption efficiency and resolution.

Semiconductor radiation detectors largely overcome this problem by direct conversion of radiation into electric signals. A semiconductor radiation detector may include a semiconductor layer that absorbs radiation in wavelengths of interest. When a particle of radiation is absorbed in the semiconductor layer, multiple charge carriers (e.g., electrons and holes) are generated and swept under an electric field towards electric contacts on the semiconductor layer. Cumbersome heat management required in currently available semiconductor radiation detectors (e.g., Medipix) can make a detector with a large area and a large number of pixels difficult or impossible to produce.

### SUMMARY

Disclosed herein is an apparatus comprising: a radiation detector; a collimator; wherein the collimator and the radiation detector are configured to collectively translate relative to a radiation source along a direction without relative movement the collimator and the radiation detector; wherein the collimator comprises a plurality of planar plates parallel to one another; and wherein the planar plates are not parallel to the direction.

According to an embodiment, an angle between the planar plates and the direction is smaller than 5 degrees, 10 degrees, 25 degrees, or 45 degrees.

According to an embodiment, the planar plates are perpendicular to a radiation receiving surface of the radiation detector.

According to an embodiment, the collimator and the radiation detector are configured to collectively move to a plurality of positions relative to the radiation source by translating along the direction relative to the radiation source.

According to an embodiment, the radiation detector is configured to capture images of portions of a scene at the plurality of positions.

According to an embodiment, the apparatus is configured to form an image of the scene by stitching the images of portions.

According to an embodiment, the radiation detector comprises an array of pixels.

According to an embodiment, the radiation detector is rectangular in shape.

According to an embodiment, the radiation detector is hexagonal in shape.

According to an embodiment, the radiation detector is configured to count numbers of particles of radiation incident on the pixels, within a period of time.

According to an embodiment, the particles of radiation are photons of X-ray.

According to an embodiment, the radiation detector comprises: a radiation absorption layer comprising an electric contact; a first voltage comparator configured to compare a voltage of the electric contact to a first threshold; a second voltage comparator configured to compare the voltage to a second threshold; a counter configured to register at least one of the numbers; a controller; wherein the controller is configured to start a time delay from a time at which the first voltage comparator determines that an absolute value of the voltage equals or exceeds an absolute value of the first threshold; wherein the controller is configured to activate the second voltage comparator during the time delay; wherein the controller is configured to cause at least one of the numbers to increase by one, when the second voltage comparator determines that an absolute value of the voltage equals or exceeds an absolute value of the second threshold.

According to an embodiment, the apparatus further comprises an integrator electrically connected to the electric contact, wherein the integrator is configured to collect charge carriers from the electric contact.

According to an embodiment, the controller is configured to activate the second voltage comparator at a beginning or expiration of the time delay.

According to an embodiment, the controller is configured to connect the electric contact to an electrical ground.

According to an embodiment, a rate of change of the voltage is substantially zero at expiration of the time delay.

According to an embodiment, the radiation detector does not comprise a scintillator.

### BRIEF DESCRIPTION OF FIGURES

FIG. 1A schematically show a perspective view of an apparatus translating along a direction, according to an embodiment.

FIG. 1B schematically shows a top view of a portion of the apparatus translating along the direction, according to an embodiment.

FIG. 2 schematically shows the apparatus capturing a plurality of images of portions of a scene, according to an embodiment.

FIG. 3A-FIG. 3C schematically show arrangements of radiation detectors in the apparatus, according to some embodiments.

FIG. 4 schematically shows an apparatus with plurality of radiation detectors that are hexagonal in shape, according to an embodiment.

FIG. 5 schematically shows that the radiation detector has an array of pixels, according to an embodiment.

FIG. 6A schematically shows a cross-sectional view of a radiation detector, according to an embodiment.

FIG. 6B schematically shows a detailed cross-sectional view of the radiation detector, according to an embodiment.

FIG. 6C schematically shows an alternative detailed cross-sectional view of the radiation detector, according to an embodiment.

FIG. 7A and FIG. 7B each show a component diagram of an electronic system of the radiation detector in FIG. 4A, FIG. 4B and FIG. 4C, according to an embodiment.

FIG. 8 schematically shows a temporal change of the electric current flowing through an electrode (upper curve) of a diode or an electric contact of a resistor of a radiation absorption layer exposed to radiation, the electric current caused by charge carriers generated by a particle of radiation incident on the radiation absorption layer, and a corresponding temporal change of the voltage of the electrode (lower curve), according to an embodiment.

### DETAILED DESCRIPTION

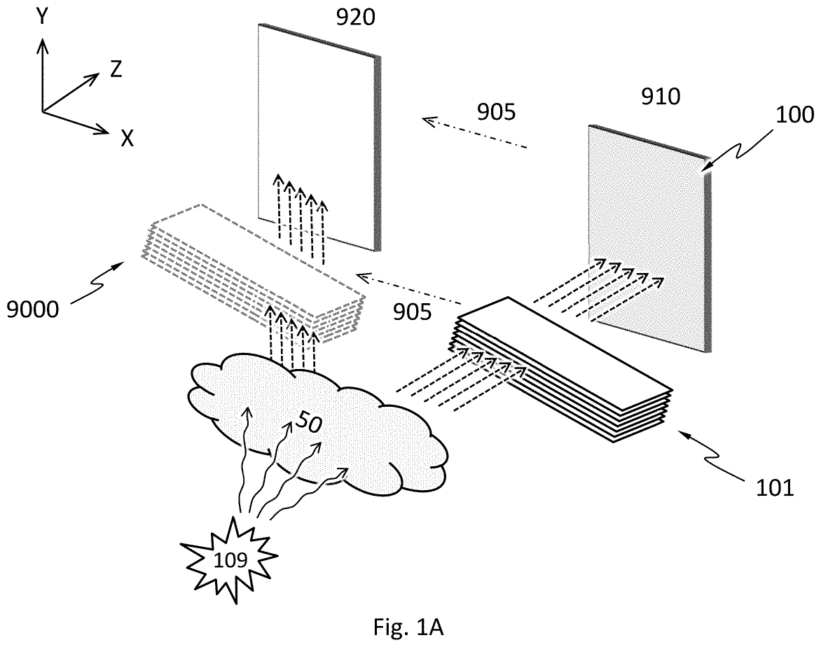

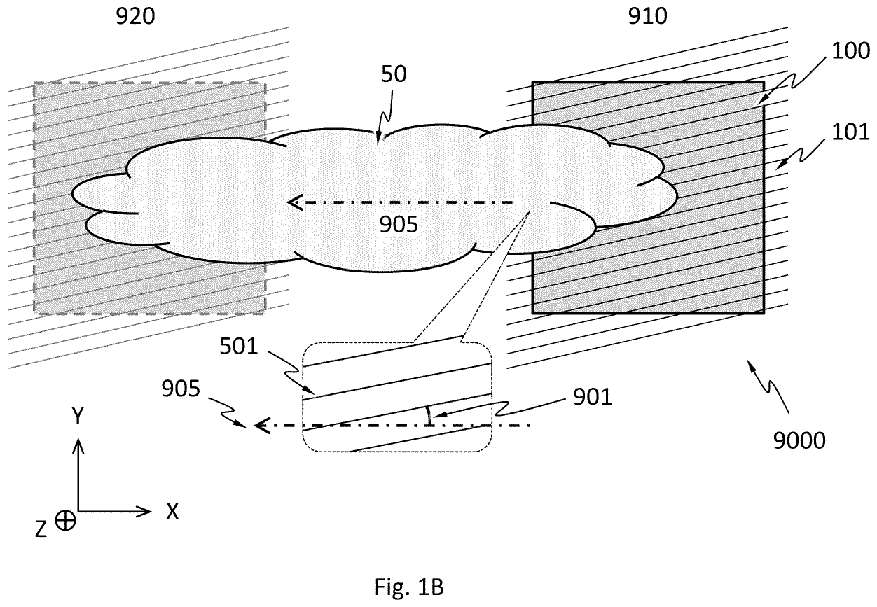

FIG. 1A schematically shows a perspective view of an apparatus 9000 translating along a direction 905, according to an embodiment. The apparatus 9000 may include a radiation detector 100 and a collimator 101. The collimator 101 may be positioned between the radiation detector 100 and a radiation source 109. The collimator 101 may include a plurality of planar plates parallel to one another (e.g., planar plates 501 in FIG. 1B). In an example, radiation from the radiation source 109 reaches a scene 50 (e.g., portions of a human body) before passing through the collimator 101 and reaching the radiation detector 100, as shown in FIG. 1A. In an example, the radiation incident on the scene 50 may partially transmit through the scene 50. The collimator 101 is configured to allow the transmitted portion of the radiation to reach the radiation detector 100 and to essentially prevent portions of the radiation scattered by the scene 50 to reach the radiation detector 100.

In an embodiment, the collimator 101 and the radiation detector 100 are configured to collectively translate relative to the radiation source 109 along a direction 905 without relative movement between the collimator 101 and the radiation detector 100. In the example shown in FIG. 1A, the collimator 101 and the radiation detector 100 may collectively translate to a plurality of positions along the direction 905, e.g., a first position 910, a second position 920, relative to the radiation source 109. During translation and at the plurality of positions, the collimator 101 may remain stationary relative to the radiation detector 100. In one embodiment, a plurality of sets of images of portions of the scene 50 are captured when the radiation detector 100 and the collimator 101 are collectively at the plurality of positions along the direction 905 relative to the radiation source 109, respectively.

FIG. 1B schematically shows a top view of a portion of the apparatus 9000 translating along the direction 905, according to an embodiment. As shown in FIG. 1B, the planar plates 501 of the collimator 101 may be perpendicular to a radiation receiving surface of the radiation detector 100. In one embodiment, the planar plates 501 are not parallel to the direction 905 but are at a small angle 901 relative to the direction 905. In an embodiment, the planar plates 501 are not perpendicular to the direction 905. The angle 901 may be smaller than 5 degrees, 10 degrees, 25 degrees, or 45 degrees.

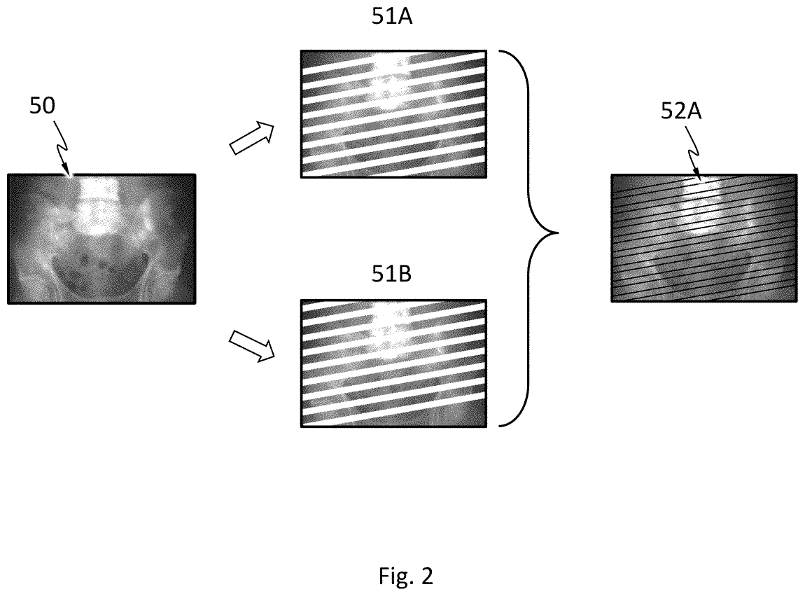

FIG. 2 schematically shows the apparatus 9000 capturing a plurality of images of portions of the scene 50, according to an embodiment. In the example shown in FIG. 1A and FIG. 1B, the radiation detector 100 and the collimator 101 may collectively translate to 2 positions 910 and 920. Respectively at the positions 910 and 920, images 51A, 51B of the portions of the scene 50 may be captured. In an example, the portion of the scene 50 in image 51A may substantially overlaps the portion of the scene 50 in image 51B. The apparatus 9000 may stitch the images 51A and 51B to form an image 52A of the scene 50. The images 51A and 51B may have overlap among them to facilitate stitching. In an example, the planar plates 501 of the collimator 101 block different parts of the scene 50 at the positions 910 and 920, which leads to the stripes in images 51A and 51B schematically shown in FIG. 2 but the positions 910 and 920 are such that the parts blocked are not identical. Every portion of the scene 50 may be in at least one of the images captured when the radiation detector 100 are at the multiple positions. Namely, the images of the portions when stitched together may cover the entire scene 50.

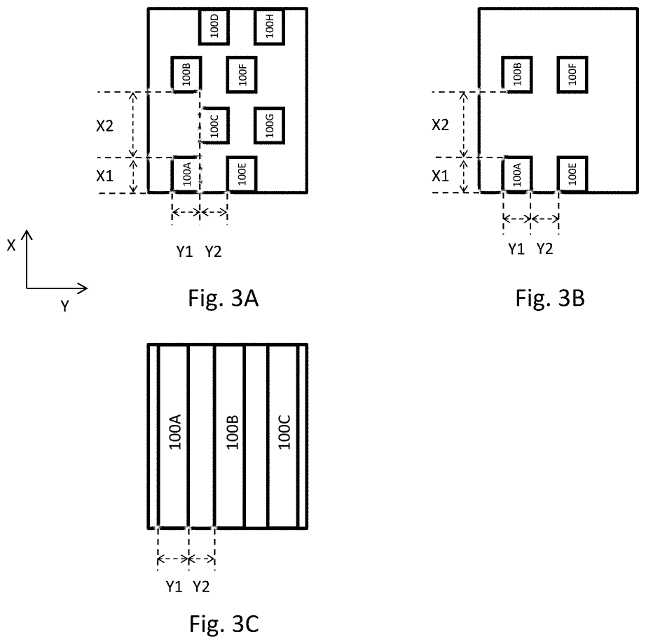

The apparatus may include multiple radiation detectors 100 arranged in a variety of ways, as shown in FIGS. 3A-3C. FIG. 3A schematically shows one arrangement, according to an embodiment, where the radiation detectors 100 are arranged in staggered rows. For example, radiation detectors 100A and 100B are in the same row, aligned in the Y direction, and uniform in size; radiation detectors 100C and 100D are in the same row, aligned in the Y direction, and uniform in size. Radiation detectors 100A and 100B are staggered in the X direction with respect to radiation detectors 100C and 100D. According to an embodiment, a distance X2 between two neighboring radiation detectors 100A and 100B in the same row is greater than a width X1 (i.e., dimension in the X direction, which is the extending direction of the row) of one radiation detector in the same row and is less than twice the width X1. Radiation detectors 100A and 100E are in a same column, aligned in the X direction, and uniform in size; a distance Y2 between two neighboring radiation detectors 100A and 100E in the same column is less than a width Y1 (i.e., dimension in the Y direction) of one radiation detector in the same column.

FIG. 3B schematically shows another arrangement, according to an embodiment, where the radiation detectors 100 are arranged in a rectangular grid. For example, the radiation detectors 100 may include radiation detectors 100A, 100B, 100E and 100F as arranged exactly in FIG. 3A, without radiation detectors 100C, 100D, 100G, or 100H in FIG. 3A. This arrangement allows imaging of the scene by taking images of portions of the scene at six positions. For example, three positions spaced apart in the X direction and another three positions spaced apart in the X direction and spaced apart in the Y direction from the first three positions.

Other arrangements may also be possible. For example, in FIG. 3C, the radiation detectors 100 may span the whole width of the X-direction, with a distance Y2 between two neighboring radiation detectors 100 being less than a width of one radiation detector Y1. Assuming the width of the detectors in the X direction is greater than the width of the scene in the X direction, the image of the scene may be stitched from two images of portions of the scene captured at two positions spaced apart in the Y direction.

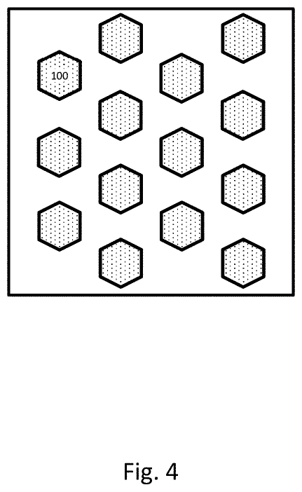

The radiation detectors 100 described above may be provided with any suitable size and shapes. According to an embodiment, at least some of the radiation detectors are rectangular in shape. According to an embodiment, as shown in FIG. 4, at least some of the radiation detectors are hexagonal in shape.

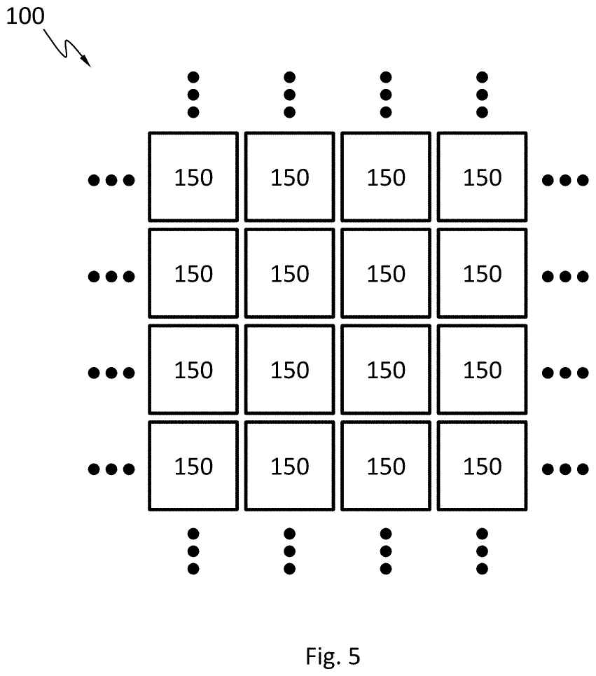

FIG. 5 schematically shows that the radiation detector 100 may have an array of pixels 150. The array may be a rectangular array, a honeycomb array, a hexagonal array or any other suitable array. Each pixel 150 may be configured to detect a particle of radiation incident thereon, measure the energy of the particle of radiation, or both. For example, each pixel 150 may be configured to count numbers of particles of radiation incident thereon whose energy falls in a plurality of bins, within a period of time. All the pixels 150 may be configured to count the numbers of particles of radiation incident thereon within a plurality of bins of energy within the same period of time. Each pixel 150 may have its own analog-to-digital converter (ADC) configured to digitize an analog signal representing the energy of an incident particle of radiation into a digital signal. The ADC may have a resolution of 10 bits or higher. Each pixel 150 may be configured to measure its dark current, such as before or concurrently with each particle of radiation incident thereon. Each pixel 150 may be configured to deduct the contribution of the dark current from the energy of the particle of radiation incident thereon. The pixels 150 may be configured to operate in parallel. For example, when one pixel 150 measures an incident particle of radiation, another pixel 150 may be waiting for another particle of radiation to arrive. The pixels 150 may be but do not have to be individually addressable. The particles of radiation may be photons of X-ray.

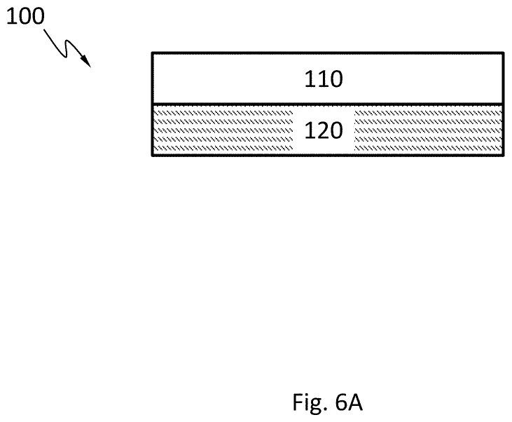

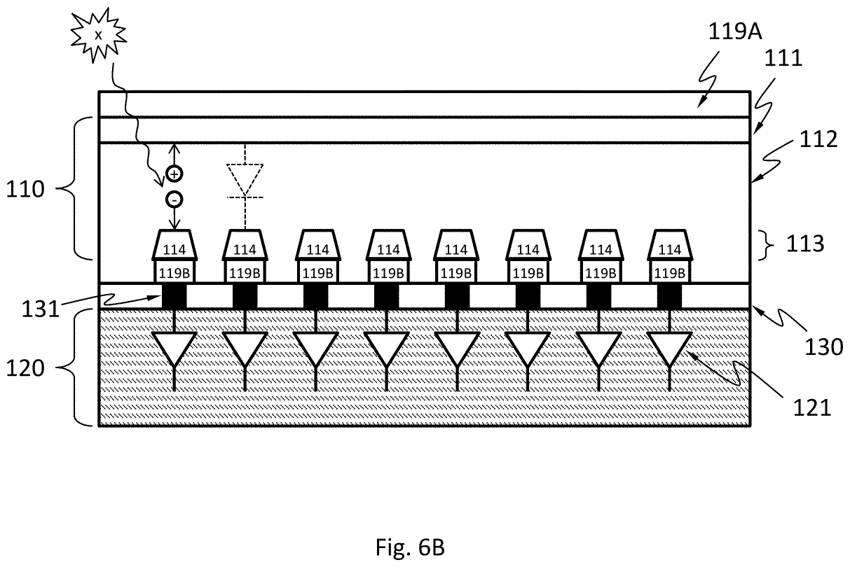

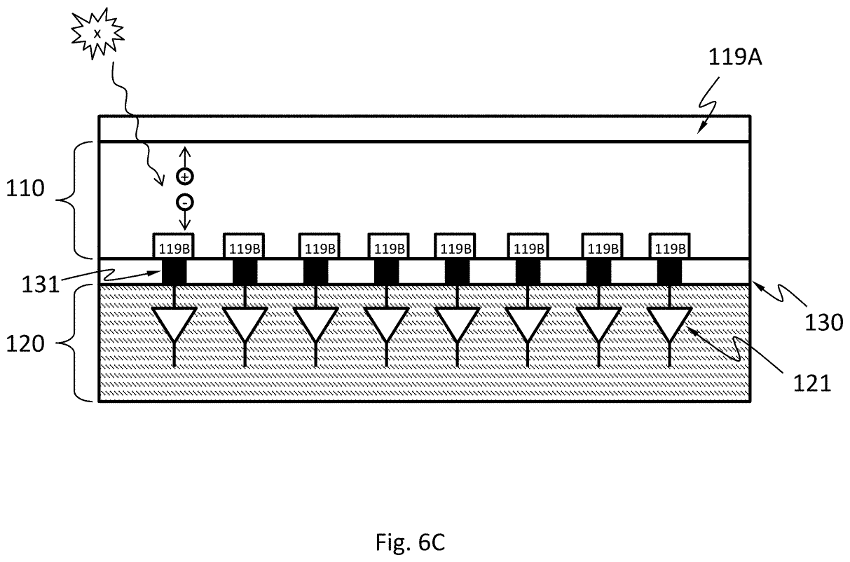

FIG. 6A schematically shows a cross-sectional view of a radiation detector 100, according to an embodiment. The radiation detector 100 may include a radiation absorption layer 110 and an electronics layer 120 (e.g., an ASIC) for processing or analyzing electrical signals incident radiation generates in the radiation absorption layer 110. In an embodiment, the radiation detector 100 does not comprise a scintillator. The radiation absorption layer 110 may include a semiconductor material such as, silicon, germanium, GaAs, CdTe, CdZnTe, or a combination thereof. The semiconductor may have a high mass attenuation coefficient for the radiation energy of interest. The surface 103 of the radiation absorption layer 110 distal from the electronics layer 120 is configured to receive radiation.

As shown in a detailed cross-sectional view of the radiation detector 100 in FIG. 6B, according to an embodiment, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional the intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example in FIG. 6B, each of the discrete regions 114 of the second doped region 113 forms a diode with the first doped region 111 and the optional intrinsic region 112. Namely, in the example in FIG. 6B, the radiation absorption layer 110 has a plurality of diodes having the first doped region 111 as a shared electrode. The first doped region 111 may also have discrete portions.

When a particle of radiation hits the radiation absorption layer 110 including diodes, the particle of radiation may be absorbed and generate one or more charge carriers by a number of mechanisms. A particle of radiation may generate 10 to 100000 charge carriers. The charge carriers may drift to the electrodes of one of the diodes under an electric field. The field may be an external electric field. The electric contact 119B may include discrete portions each of which is in electrical contact with the discrete regions 114. In an embodiment, the charge carriers may drift in directions such that the charge carriers generated by a single particle of radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of radiation incident around the footprint of one of these discrete regions 114 are not substantially shared with another of these discrete regions 114. The pixel 150 associated with a discrete region 114 may be an area around the discrete region 114 in which substantially all (more than 98%, more than 99.5%, more than 99.9%, or more than 99.99% of) charge carriers generated by a particle of radiation incident therein at an angle of incidence of 0° flow to the discrete region 114. Namely, less than 2%, less than 1%, less than 0.1%, or less than 0.01% of these charge carriers flow beyond the pixel.

As shown in an alternative detailed cross-sectional view of the radiation detector 100 in FIG. 6C, according to an embodiment, the radiation absorption layer 110 may include a resistor of a semiconductor material such as, silicon, germanium, GaAs, CdTe, CdZnTe, or a combination thereof, but does not include a diode. The semiconductor may have a high mass attenuation coefficient for the radiation energy of interest.

When a particle of radiation hits the radiation absorption layer 110 including a resistor but not diodes, it may be absorbed and generate one or more charge carriers by a number of mechanisms. A particle of radiation may generate 10 to 100000 charge carriers. The charge carriers may drift to the electric contacts 119A and 119B under an electric field. The field may be an external electric field. The electric contact 119B includes discrete portions. In an embodiment, the charge carriers may drift in directions such that the charge carriers generated by a single particle of radiation are not substantially shared by two different discrete portions of the electric contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of radiation incident around the footprint of one of these discrete portions of the electric contact 119B are not substantially shared with another of these discrete portions of the electric contact 119B. The pixel 150 associated with a discrete portion of the electric contact 119B may be an area around the discrete portion in which substantially all (more than 98%, more than 99.5%, more than 99.9% or more than 99.99% of) charge carriers generated by a particle of radiation incident at an angle of incidence of 0° therein flow to the discrete portion of the electric contact 119B. Namely, less than 2%, less than 0.5%, less than 0.1%, or less than 0.01% of these charge carriers flow beyond the pixel associated with the one discrete portion of the electric contact 119B.

The electronics layer 120 may include an electronic system 121 suitable for processing or interpreting signals generated by particles of radiation incident on the Radiation absorption layer 110. The electronic system 121 may include an analog circuitry such as a filter network, amplifiers, integrators, and comparators, or a digital circuitry such as a microprocessor, and memory. The electronic system 121 may include components shared by the pixels or components dedicated to a single pixel. For example, the electronic system 121 may include an amplifier dedicated to each pixel and a microprocessor shared among all the pixels. The electronic system 121 may be electrically connected to the pixels by vias 131. Space among the vias may be filled with a filler material 130, which may increase the mechanical stability of the connection of the electronics layer 120 to the radiation absorption layer 110. Other bonding techniques are possible to connect the electronic system 121 to the pixels without using vias.

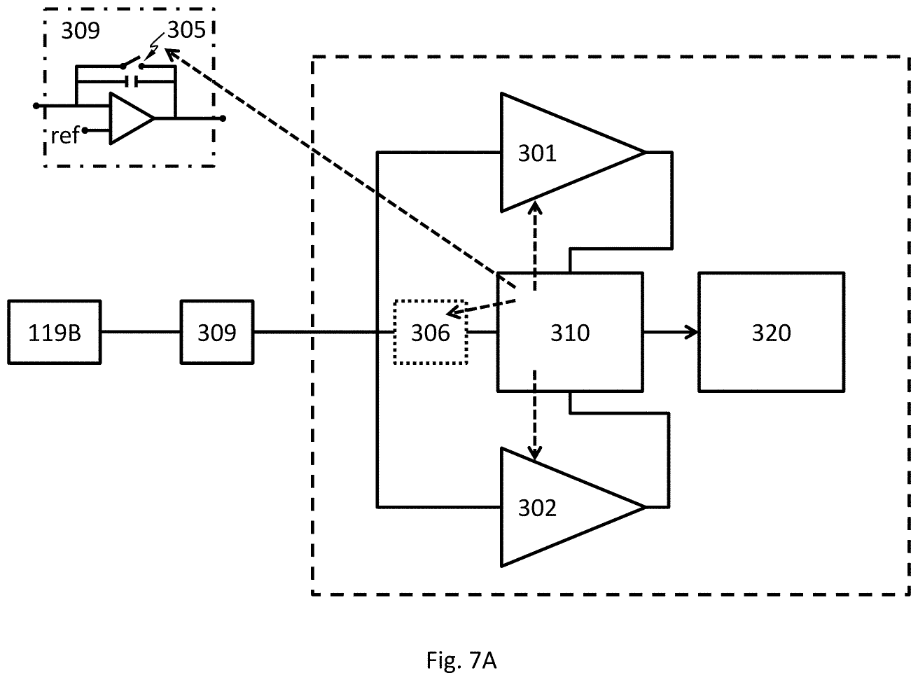

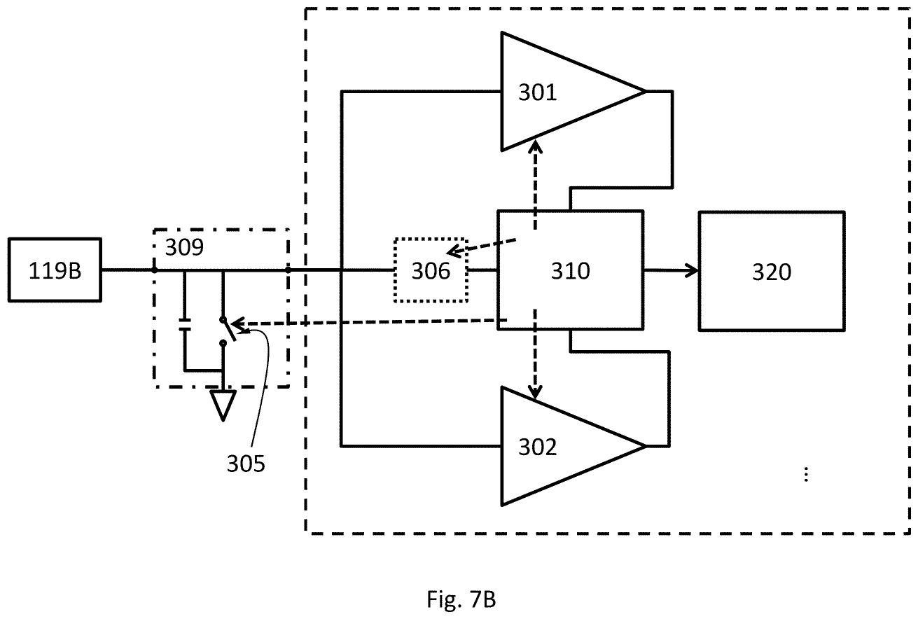

FIG. 7A and FIG. 7B each show a component diagram of the electronic system 121, according to an embodiment. The electronic system 121 may include a first voltage comparator 301, a second voltage comparator 302, a counter 320, a switch 305, an optional voltmeter 306 and a controller 310.

The first voltage comparator 301 is configured to compare the voltage of at least one of the electric contacts 119B to a first threshold. The first voltage comparator 301 may be configured to monitor the voltage directly, or calculate the voltage by integrating an electric current flowing through the electrical contact 119B over a period of time. The first voltage comparator 301 may be controllably activated or deactivated by the controller 310. The first voltage comparator 301 may be a continuous comparator. Namely, the first voltage comparator 301 may be configured to be activated continuously and monitor the voltage continuously. The first voltage comparator 301 may be a clocked comparator. The first threshold may be 5-10%, 10%-20%, 20-30%, 30-40% or 40-50% of the maximum voltage one incident particle of radiation may generate on the electric contact 119B. The maximum voltage may depend on the energy of the incident particle of radiation, the material of the radiation absorption layer 110, and other factors. For example, the first threshold may be 50 mV, 100 mV, 150 mV, or 200 mV.

The second voltage comparator 302 is configured to compare the voltage to a second threshold. The second voltage comparator 302 may be configured to monitor the voltage directly or calculate the voltage by integrating an electric current flowing through the diode or the electrical contact over a period of time. The second voltage comparator 302 may be a continuous comparator. The second voltage comparator 302 may be controllably activate or deactivated by the controller 310. When the second voltage comparator 302 is deactivated, the power consumption of the second voltage comparator 302 may be less than 1%, less than 5%, less than 10% or less than 20% of the power consumption when the second voltage comparator 302 is activated. The absolute value of the second threshold is greater than the absolute value of the first threshold. As used herein, the term “absolute value” or “modulus” |x| of a real number x is the non-negative value of x without regard to its sign. Namely,

$$\{ \begin{matrix}x , if x \geq 0 \\ - x , if x \leq 0\end{matrix} .$$

The second threshold may be 200%-300% of the first threshold. The second threshold may be at least 50% of the maximum voltage one incident particle of radiation may generate on the electric contact 119B. For example, the second threshold may be 100 mV, 150 mV, 200 mV, 250 mV or 300 mV. The second voltage comparator 302 and the first voltage comparator 301 may be the same component. Namely, the system 121 may have one voltage comparator that can compare a voltage with two different thresholds at different times.

The first voltage comparator 301 or the second voltage comparator 302 may include one or more op-amps or any other suitable circuitry. The first voltage comparator 301 or the second voltage comparator 302 may have a high speed to allow the electronic system 121 to operate under a high flux of incident particles of radiation. However, having a high speed is often at the cost of power consumption.

The counter 320 is configured to register at least a number of particles of radiation incident on the pixel 150 encompassing the electric contact 119B. The counter 320 may be a software component (e.g., a number stored in a computer memory) or a hardware component (e.g., a 4017 IC and a 7490 IC).

The controller 310 may be a hardware component such as a microcontroller and a microprocessor. The controller 310 is configured to start a time delay from a time at which the first voltage comparator 301 determines that the absolute value of the voltage equals or exceeds the absolute value of the first threshold (e.g., the absolute value of the voltage increases from below the absolute value of the first threshold to a value equal to or above the absolute value of the first threshold). The absolute value is used here because the voltage may be negative or positive, depending on whether the voltage of the cathode or the anode of the diode or which electrical contact is used. The controller 310 may be configured to keep deactivated the second voltage comparator 302, the counter 320 and any other circuits the operation of the first voltage comparator 301 does not require, before the time at which the first voltage comparator 301 determines that the absolute value of the voltage equals or exceeds the absolute value of the first threshold. The time delay may expire before or after the voltage becomes stable, i.e., the rate of change of the voltage is substantially zero. The phase “the rate of change of the voltage is substantially zero” means that temporal change of the voltage is less than 0.1%/ns. The phase “the rate of change of the voltage is substantially non-zero” means that temporal change of the voltage is at least 0.1%/ns.

The controller 310 may be configured to activate the second voltage comparator during (including the beginning and the expiration) the time delay. In an embodiment, the controller 310 is configured to activate the second voltage comparator at the beginning of the time delay. The term “activate” means causing the component to enter an operational state (e.g., by sending a signal such as a voltage pulse or a logic level, by providing power, etc.). The term “deactivate” means causing the component to enter a non-operational state (e.g., by sending a signal such as a voltage pulse or a logic level, by cut off power, etc.). The operational state may have higher power consumption (e.g., 10 times higher, 100 times higher, 1000 times higher) than the non-operational state. The controller 310 itself may be deactivated until the output of the first voltage comparator 301 activates the controller 310 when the absolute value of the voltage equals or exceeds the absolute value of the first threshold.

The controller 310 may be configured to cause at least one of the number registered by the counter 320 to increase by one, if, during the time delay, the second voltage comparator 302 determines that the absolute value of the voltage equals or exceeds the absolute value of the second threshold.

The controller 310 may be configured to cause the optional voltmeter 306 to measure the voltage upon expiration of the time delay. The controller 310 may be configured to connect the electric contact 119B to an electrical ground, so as to reset the voltage and discharge any charge carriers accumulated on the electric contact 119B. In an embodiment, the electric contact 119B is connected to an electrical ground after the expiration of the time delay. In an embodiment, the electric contact 119B is connected to an electrical ground for a finite reset time period. The controller 310 may connect the electric contact 119B to the electrical ground by controlling the switch 305. The switch may be a transistor such as a field-effect transistor (FET).

In an embodiment, the system 121 has no analog filter network (e.g., a RC network). In an embodiment, the system 121 has no analog circuitry.

The voltmeter 306 may feed the voltage it measures to the controller 310 as an analog or digital signal.

The electronic system 121 may include an integrator 309 electrically connected to the electric contact 119B, wherein the integrator is configured to collect charge carriers from the electric contact 119B. The integrator 309 can include a capacitor in the feedback path of an amplifier. The amplifier configured as such is called a capacitive transimpedance amplifier (CTIA). CTIA has high dynamic range by keeping the amplifier from saturating and improves the signal-to-noise ratio by limiting the bandwidth in the signal path. Charge carriers from the electric contact 119B accumulate on the capacitor over a period of time (“integration period”). After the integration period has expired, the capacitor voltage is sampled and then reset by a reset switch. The integrator 309 may include a capacitor directly connected to the electric contact 119B.

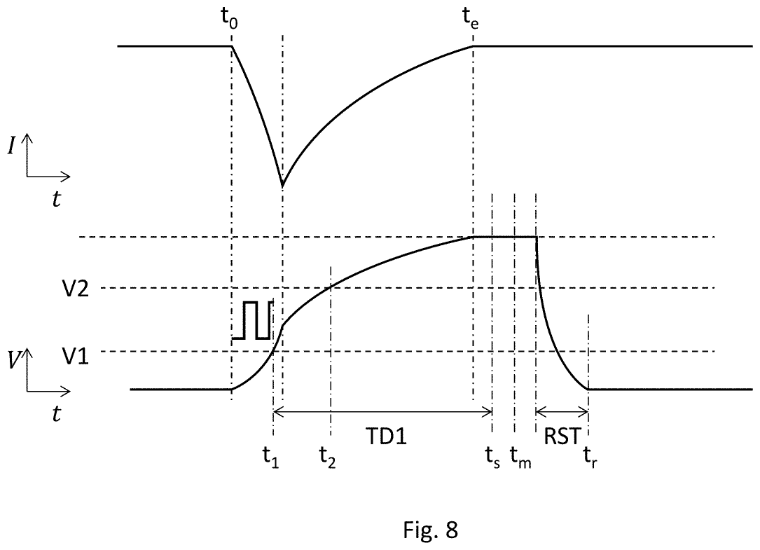

FIG. 8 schematically shows a temporal change of the electric current flowing through the electric contact 119B (upper curve) caused by charge carriers generated by a particle of radiation incident on the pixel 150 encompassing the electric contact 119B, and a corresponding temporal change of the voltage of the electric contact 119B (lower curve). The voltage may be an integral of the electric current with respect to time. At time t₀, the particle of radiation hits pixel 150, charge carriers start being generated in the pixel 150, electric current starts to flow through the electric contact 119B, and the absolute value of the voltage of the electric contact 119B starts to increase. At time t₁, the first voltage comparator 301 determines that the absolute value of the voltage equals or exceeds the absolute value of the first threshold V1, and the controller 310 starts the time delay TD1 and the controller 310 may deactivate the first voltage comparator 301 at the beginning of TD1. If the controller 310 is deactivated before t₁, the controller 310 is activated at t₁. During TD1, the controller 310 activates the second voltage comparator 302. The term “during” a time delay as used here means the beginning and the expiration (i.e., the end) and any time in between. For example, the controller 310 may activate the second voltage comparator 302 at the expiration of TD1. If during TD1, the second voltage comparator 302 determines that the absolute value of the voltage equals or exceeds the absolute value of the second threshold V2 at time t₂, the controller 310 waits for stabilization of the voltage to stabilize. The voltage stabilizes at time tₑ, when all charge carriers generated by the particle of radiation drift out of the radiation absorption layer 110. At time t₅, the time delay TD1 expires. At or after time tₑ, the controller 310 causes the voltmeter 306 to digitize the voltage and determines which bin the energy of the particle of radiation falls in. The controller 310 then causes the number registered by the counter 320 corresponding to the bin to increase by one. In the example of FIG. 8, time tₛ is after time tₑ; namely TD1 expires after all charge carriers generated by the particle of radiation drift out of the radiation absorption layer 110. If time tₑ cannot be easily measured, TD1 can be empirically chosen to allow sufficient time to collect essentially all charge carriers generated by a particle of radiation but not too long to risk have another incident particle of radiation. Namely, TD1 can be empirically chosen so that time tₛ is empirically after time tₑ. Time tₛ is not necessarily after time tₑ because the controller 310 may disregard TD1 once V2 is reached and wait for time tₑ. The rate of change of the difference between the voltage and the contribution to the voltage by the dark current is thus substantially zero at tₑ. The controller 310 may be configured to deactivate the second voltage comparator 302 at expiration of TD1 or at t₂, or any time in between.

The voltage at time tₑ is proportional to the amount of charge carriers generated by the particle of radiation, which relates to the energy of the particle of radiation. The controller 310 may be configured to determine the energy of the particle of radiation, using the voltmeter 306.

After TD1 expires or digitization by the voltmeter 306, whichever later, the controller 310 connects the electric contact 119B to an electric ground for a reset period RST to allow charge carriers accumulated on the electric contact 119B to flow to the ground and reset the voltage. After RST, the electronic system 121 is ready to detect another incident particle of radiation. If the first voltage comparator 301 has been deactivated, the controller 310 can activate it at any time before RST expires. If the controller 310 has been deactivated, it may be activated before RST expires.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An apparatus comprising:
a radiation detector;
a collimator;
wherein the collimator and the radiation detector are configured to collectively translate relative to a radiation source along a direction without relative movement between the collimator and the radiation detector;
wherein the collimator comprises a plurality of planar plates parallel to one another; and
wherein the planar plates are not parallel to the direction.

2. The apparatus of claim 1, wherein an angle between the planar plates and the direction is smaller than 5 degrees, 10 degrees, 25 degrees, or 45 degrees.

3. The apparatus of claim 1, wherein the planar plates are perpendicular to a radiation receiving surface of the radiation detector.

4. The apparatus of claim 1, wherein the collimator and the radiation detector are configured to collectively move to a plurality of positions by translating along the direction relative to the radiation source.

5. The apparatus of claim 4, wherein the apparatus is configured to capture images of portions of a scene at the plurality of positions.

6. The apparatus of claim 5, wherein the apparatus is configured to form an image of the scene by stitching the images of portions.

7. The apparatus of claim 1, wherein the radiation detector comprises an array of pixels.

8. The apparatus of claim 1, wherein the radiation detector is rectangular in shape.

9. The apparatus of claim 1, wherein the radiation detector is hexagonal in shape.

10. The apparatus of claim 7, wherein the radiation detector is configured to count numbers of particles of radiation incident on the pixels, within a period of time.

11. The apparatus of claim 10, wherein the particles of radiation are photons of X-ray.

12. The apparatus of claim 10, wherein the radiation detector comprises:
a radiation absorption layer comprising an electric contact;
a first voltage comparator configured to compare a voltage of the electric contact to a first threshold;
a second voltage comparator configured to compare the voltage to a second threshold;
a counter configured to register at least one of the numbers;
a controller;
wherein the controller is configured to start a time delay from a time at which the first voltage comparator determines that an absolute value of the voltage equals or exceeds an absolute value of the first threshold;
wherein the controller is configured to activate the second voltage comparator during the time delay;
wherein the controller is configured to cause at least one of the numbers to increase by one, when the second voltage comparator determines that an absolute value of the voltage equals or exceeds an absolute value of the second threshold.

13. The apparatus of claim 12, further comprising an integrator electrically connected to the electric contact, wherein the integrator is configured to collect charge carriers from the electric contact.

14. The apparatus of claim 12, wherein the controller is configured to activate the second voltage comparator at a beginning or expiration of the time delay.

15. The apparatus of claim 12, wherein the controller is configured to connect the electric contact to an electrical ground.

16. The apparatus of claim 12, wherein a rate of change of the voltage is substantially zero at expiration of the time delay.

17. The apparatus of claim 1, wherein the radiation detector does not comprise a scintillator.

18. The apparatus of claim 1, wherein the planar plates are not perpendicular to the direction.
